SEQUENCE LISTING (7 sequences)

SEQ ID NO: 1 - protein, 28 aa, Artificial Sequence
  28-residue 1
NYYIHWVKQAPGKSLEWIGQHYAGYSAT

SEQ ID NO: 2 - protein, 28 aa, Artificial Sequence
  28-residue 2
SYAMHWVRQAPGKGLEWVAWDSSLSAVV

SEQ ID NO: 3 - protein, 625 aa, Artificial Sequence
  colicin Ia
SDPVRITNPGAESLGYDSDGHEIMAVDIYVNPPRVDVFHGTPPAWSSFGNKTIWGGNEWVDDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVAEWDKLRQRLLDARNKITSAESAVNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVRTENWRPLFVKTETIIAGNAATALVALVFSILTGSALGIIGYGLLMAVTGALIDESLVEKANKFWGI

SEQ ID NO: 4 - protein, 653 aa, Artificial Sequence
  pheromonicin-covid-19-E
SDPVRITNPGAESLGYDSDGHEIMAVDIYVNPPRVDVFHGTPPAWSSFGNKTIWGGNEWVDDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVAEWDKLRQRLLDARNKITSAESAVNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVRTENWRPLFVKTETIIAGNAATALVALVFSILTGSALGIIGYGLLMAVTGALIDESLVEKANKFWGINYYIHWVKQAPGKSLEWIGQHYAGYSAT

SEQ ID NO: 5 - protein, 653 aa, Artificial Sequence
  pheromonicin-covid-19-M
SDPVRITNPGAESLGYDSDGHEIMAVDIYVNPPRVDVFHGTPPAWSSFGNKTIWGGNEWVDDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVAEWDKLRQRLLDARNKITSAESAVNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVRTENWRPLFVKTETIIAGNAATALVALVFSILTGSALGIIGYGLLMAVTGALIDESLVEKANKFWGISYAMHWVRQAPGKGLEWVAWDSSLSAVV

SEQ ID NO: 6 - protein, 681 aa, Artificial Sequence
  pheromonicin-covid-19-E/M
SDPVRITNPGAESLGYDSDGHEIMAVDIYVNPPRVDVFHGTPPAWSSFGNKTIWGGNEWVDDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVAEWDKLRQRLLDARNKITSAESAVNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVRTENWRPLFVKTETIIAGNAATALVALVFSILTGSALGIIGYGLLMAVTGALIDESLVEKANKFWGINYYIHWVKQAPGKSLEWIGQHYAGYSATSYAMHWVRQAPGKGLEWVAWDSSLSAVV

SEQ ID NO: 7 - protein, 681 aa, Artificial Sequence
  pheromonicin-covid-19-M/E
SDPVRITNPGAESLGYDSDGHEIMAVDIYVNPPRVDVFHGTPPAWSSFGNKTIWGGNEWVDDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVAEWDKLRQRLLDARNKITSAESAVNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVRTENWRPLFVKTETIIAGNAATALVALVFSILTGSALGIIGYGLLMAVTGALIDESLVEKANKFWGISYAMHWVRQAPGKGLEWVAWDSSLSAVVNYYIHWVKQAPGKSLEWIGQHYAGYSAT

(19) United States
(12) Patent Application Publication
(10) Pub. No.: US 2025/0319181 A1
(43) Pub. Date: Oct. 16, 2025

(54) PHEROMONICIN AGAINST SARS-COV-2 AND USE THEREOF

(71) Applicant: PHEROMONICIN BIOTECHNOLOGY LIMITED, Beijing (CN)

(72) Inventor: Xiaoqing QIU, Beijing (CN)

(73) Assignee: PHEROMONICIN BIOTECHNOLOGY LIMITED, Beijing (CN)

(21) Appl. No.: 18/694,766

(22) PCT Filed: Jan. 25, 2022
(86) PCT No.: PCT/CN2022/073668

§ 371 Date: Mar. 22, 2024

(30) Foreign Application Priority Data
Sep. 28, 2021 (CN) 202111141279.1
Dec. 13, 2021 (CN) 202111515678.X

(51) Int. Cl. A61K 39/42; A61K 38/16; A61K 39/00; A61P 31/14; C07K 14/245; C07K 16/10

(52) U.S. Cl. CPC A61K 39/42; A61K 38/164; A61P 31/14; C07K 14/245; C07K 16/1003; A61K 2039/505; C07K 2317/565; C07K 2319/00

(57) ABSTRACT

A pheromonicin against SARS-COV-2. Antibody mimetics, i.e., two 28-residues are designed for the first time by selecting the E protein and M protein of SARS-COV-2, which are relatively conserved and have low probability of mutation, as targets. Pharmacodynamic experiments performed using three SARS-COV-2 strains (the epidemic strain GD108, the South Africa strain SA and the India strain IND) respectively prove that fusion proteins obtained by linking the 28-residues to colicin can provide effective protective efficacy against pulmonary lesions induced by SARS-COV-2, and can be used as drugs for treating and preventing SARS-COV-2.

### PRIORITY APPLICATIONS

The present application is a U.S. national phase filing of International Patent Application Serial No. PCT/CN2022/073668, filed on Jan. 25, 2022, entitled “PHEROMONICINS AGAINST SARS-COV-2 AND USE THEREOF,” which application claims priority of Chinese patent application No. 202111141279.1, filed on Sep. 28, 2021, and Chinese patent application No. 202111515678.X, filed on Dec. 13, 2021. The contents and disclosures of the above-referenced applications are incorporated herein by reference in their entireties for all purposes.

### REFERENCE TO A SEQUENCE LISTING

This application contains references to nucleic acid sequences and/or amino acid sequences which have been submitted concurrently herewith as the computer readable sequence listing text file in ST.25 format: file name: Phero_ST25.txt, date recorded: Sep. 30, 2024, size: 29,695 bytes. The afore-mentioned sequence listing is hereby incorporated by reference in its entirety pursuant to 37 C.F.R. § 1.52(e)(5).

### TECHNICAL FIELD

The disclosure belongs to the field of biomedicine, and relates to drugs against SARS-CoV-2, including pheromonicins against SARS-COV-2, antibody mimetics, and a preparation method and use thereof.

### BACKGROUND

Since 2019, Severe Acute Respiratory Syndrome Corona Virus 2 (SARS-COV-2) infections have posed a huge threat to global public health. Therefore, there is an urgent need for diagnosis, prevention (vaccine) and drugs against SARS-COV-2.

At present, most of the reported candidate drugs against SARS-COV-2 are chemical drugs which have a screening effect on the virus when used, while the virus reproduces and varies rapidly with high mutation probability and high potential for development of resistance.

In the field of biopharmaceuticals, the current research and development of vaccines and drugs for the treatment of SARS-COV-2 mostly focus on the S protein of SARS-COV-2 as a target. However, it is well known that the S protein of SARS-COV-2 has high probability of mutation. Once a variation occurs, the virus variant cannot be correctly recognized and killed.

In addition, vaccines can take effect only after antibodies are produced (in several tens of days), and have no efficacy against mutated virus strains. The drugs in the prior art have no killing activity on infected host cells, and cannot alleviate inflammatory cytokine storm or alleviate toxic lesions caused by inflammation.

Therefore, there is an urgent need for drugs that have strong killing activity on SARS-CoV-2 and infected host cells, can be used and take effect immediately post infection, and have a broad spectrum against SARS-COV-2 (that is, with inhibitory activity on not only epidemic but also mutant strains of virus).

Colicins are classic specimens of bacteriocins. There are more than 20 types of colicins, which attack the genes and protein synthesis systems of other E. coli strains, or destroy the cell membrane of E. coli. Channel-forming E1 family colicins, which can form ion channels in the cell membrane to kill E. coli, include colicin E1, colicin Ia, colicin Ib, colicin A, colicin B, colicin N and the like.

The channel-forming E1 family colicins E1, Ia, Ib, A, B, N and the like are one of regulatory forces that maintain the diversity and evolution of the intestinal flora. The bacteria-killing principle of the channel-forming E1 family colicins is that, taking colicin Ia, which usually has three structural domains: a translocation domain, a receptor domain and a channel-forming domain, as an example, the channel-forming domain can form a voltage-activated ion channel in the bacterial cell membrane (lipid bilayer). The channel-forming domain of colicin Ia located at the carboxyl terminal comprises 175 amino acids and 10 a helices. Driven by hydrophilic and hydrophobic forces, the channel-forming domain can insert into the inner membrane (cell membrane) of E. coli to form an ion channel without consuming energy. The channel will open when sensing a transmembrane potential of −50 mv. Due to the large pore size (approximately 9-11 Å) of the channel, almost all ions can leak out through the huge aqueous pore, leading to depletion of energy and ion reserves of the bacteria, membrane rupture, leakage of cellular contents, and death of E. coli. The bacteria-killing process is a physical process that can achieve the bacteria-killing purpose without changing or affecting enzymes required for the growth, metabolism and reproduction of the bacteria or changing or affecting the metabolism thereof. Therefore, for hundreds of millions of years until now, the bacteria-killing process has still been effectively killing bacteria of the same species and different strains.

Colicin Ia is the type specimen of E1 family colicins, with the gene, protein structure and working mechanism which are most well understood and detailed among the E1 family colicins.

### SUMMARY

The objective of the disclosure is to provide drugs against SARS-COV-2, which can specifically recognize some surface antigens (proteins and/or hydrocarbons) of SARS-COV-2 and demonstrate protective efficacy against virus variants.

Specifically, the disclosure achieves the objective through the following works:

1. The disclosure pioneers the design of antibody mimetics (Ab Mimetics) specific for SARS-COV-2.

The antibody mimetics are selected from two types of 28-residues, with amino acid sequences of SEQ ID NO. 1 and SEQ ID NO. 2 (hereinafter referred to as 28-residue 1 and 28-residue 2).

The antibody mimetics are constructed using the antibody sequences of the disclosed E protein and M protein of SARS-COV-2 as blueprints, and can recognize the E protein and M protein of SARS-COV-2.

2. Channel-forming E1 family colicins are linked to the polypeptides with the sequence shown in SEQ ID NO. 1 and/or SEQ ID NO. 2, and the fusion proteins obtained are drugs against SARS-COV-2, i.e., pheromonicins against SARS-COV-2.

The channel-forming E1 family colicins include colicins E1, Ia, Ib, A, B and N.

The preferred colicin is colicin Ia, with an amino acid sequence shown in SEQ ID NO. 3.

The linkage is achieved by linking the 28-residue 1 and/or 28-residue 2 to the carboxyl terminal and/or amino terminal of the colicin.

Preferably, the 28-residue 1 and/or 28-residue 2 is linked to the carboxyl terminal of the colicin by a covalent bond.

When two 28-residues are linked, the preferred linkage is in a sequence as follows:
colicin-28-residue 1-28-residue 2; or
colicin-28-residue 2-28-residue 1.

That is, the pheromonicins against SARS-COV-2 are preferably the fusion proteins with the following sequences:
SEQ ID NO. 3-SEQ ID NO. 1;
SEQ ID NO. 3-SEQ ID NO. 2;
SEQ ID NO. 3-SEQ ID NO. 1-SEQ ID NO. 2; or
SEQ ID NO. 3-SEQ ID NO. 2-SEQ ID NO. 1.

Hereafter in the disclosure, the products of linkage of the antibody mimetics to the colicin are also referred to as “pheromonicins”.

The pheromonicins of the disclosure have a unique antiviral mechanism of disrupting the integrity of lipid bilayers. Using a 28-residue antibody mimetic structure constructed by a V_{H}CDR1-V_{H}FR2-V_{L}CDR3 primary structure sequence designed by the inventor, and using the disclosed antibody sequences against the E protein and M protein of SARS-COV-2 as blueprints, the disclosure constructs two 28-residue antibody mimetics capable of recognizing the E protein and M protein. The two 28-residue antibody mimetics are linked to the carboxyl terminal and amino terminal of the colicin Ia respectively, to construct 8 types of pheromonicins, which are respectively pheromonicins with the antibody mimetics linked to the carboxyl terminal of the colicin: pheromonicin-COVID19-E (PMC-E), pheromonicin-COVID19-M (PMC-M), pheromonicin-COVID19-E/M (PMC-E/M), and pheromonicin-COVID19-M/E (PMC-M/E); and pheromonicins with the antibody mimetics linked to the amino terminal of the colicin: PMC amino terminal E (E-colicin Ia), PMC amino terminal M (M-colicin Ia), PMC amino terminal E/M (E/M-colicin Ia), and PMC amino terminal M/E (M/E-colicin Ia).

The antibody mimetics are formed by linking V_{H}CDR1 (heavy chain antigen binding region 1), V_{H}FR2 (heavy chain framework region 2) and V_{L}CDR3 (light chain antigen binding region 3) selected from Fab segments of antibodies that recognize the E protein of SARS-COV-2 in a linear V_{H}CDR1-V_{H}FR2-V_{L}CDR3 primary structure sequence.

Preferably, the NCBI accession numbers of the antibodies that recognize the E protein of SARS-COV-2 are 7CWN_E and 7L06_E.

Preferably, the antibody mimetics are formed by linking V_{H}CDR1, V_{H}FR2 and V_{L}CDR3 selected from Fab segments of antibodies that recognize the M protein of SARS-COV-2 in a linear V_{H}CDR1-V_{H}FR2-V_{L}CDR3 primary structure sequence.

Preferably, the NCBI accession numbers of the antibodies that recognize the M protein of SARS-COV-2 are 7CWU_M and 7CWS_M.

The disclosure further provides a method for preparing the drugs against SARS-COV-2, where proteins with amino acid sequences shown in SEQ ID NO. 1 and/or SEQ ID NO. 2 are linked to colicins, and the fusion proteins obtained are the drugs against SARS-COV-2.

The colicins include colicins E1, Ia, Ib, A, B and N.

The preferred colicin is colicin Ia.

The drugs against SARS-COV-2 may be prepared in different dosage forms according to the requirements of clinical use by adding pharmaceutically acceptable excipients.

Beneficial effects and innovation of the disclosure:

1. channel-forming colicins are used.

The inventor has found through a lot of research that colicins can form ion channels in various lipid bilayers with different components and thicknesses, which suggests that if the inherent targeting (which can only recognize different strains of E. coli of the same species) of the colicins can be altered, the modified colicins may recognize other bacteria, fungi, envelope viruses and even eukaryotic cells, and therefore form ion channels in the envelopes or cell membranes (lipid bilayers) of the living organisms to kill them.

2. E protein and M protein are selected as targets.

Different from a lot of research and development that have focused the idea on the S protein of SARS-COV-2 as a target, the disclosure selects E protein and M protein, which are relatively conserved and have low probability of mutation, as targets. This choice has an advantage that because the variations of virus variants mostly occur on the S protein, while the E protein and M protein have fewer variations, the pheromonicins of the disclosure can still recognize the E protein and M protein of the virus variants and therefore kill the virus variants, which advantage is not possessed by the currently used vaccines and drugs.

The disclosure has fully proven by a large number of animal experiments that the aforementioned four pheromonicins (PMC-E, PMC-M, PMC-E/M and PMC-M/E) containing the antibody mimetics capable of recognizing the E protein and/or M protein of SARS-COV-2 can effectively alleviate the pathological state and reduce the pathological scores of the lung of animals infected with the epidemic strain (GD108), South Africa strain (SA) and India strain (IND, Delta strain) of SARS-COV-2.

To sum up, the pheromonicins of the disclosure have the following advantage: the pheromonicins have strong killing activity on SARS-COV-2 and viral-infected host cells. The antiviral active part of the pheromonicins of the disclosure is colicin Ia. The inventor of the disclosure has previously proven that colicin Ia can down regulate inflammatory cytokine storm, thereby alleviating toxic lesions caused by inflammation. Experiments have shown that colicin Ia can significantly reduce the peak of IL-6, IL-12, MIP-1a, MIP-1b, IL-12, KC and the like produced during inflammation (Qiu et al, Defending the homeland: Microbiome molecules provide protection to their vertabrate hosts. Future Microbiology, 15:1697-1712 (2020 Dec) doi: 10.2217/fmb-2020-0008 E pub 2020 Dec. 22).

3. As treatment drugs, the pheromonicins have protective efficacy against epidemic and mutant strains of virus, and can significantly reduce fatal pulmonary lesions caused by severe acute respiratory syndrome (SARS).

The pheromonicins of the disclosure can be used and take effect immediately as drugs, but vaccines take effect only after antibodies are produced (in several tens of days). Moreover, the pheromonicins of the disclosure have protective efficacy against epidemic and mutant strains of virus, that is, broad-spectrum resistance to SARS-COV-2. More importantly, experimental examples of the disclosure prove that the pheromonicins can significantly reduce SARS-COV-2-induced pulmonary lesions and save patients from death, which is of great significance for clinical treatment of COVID-19.

Pharmacodynamic experiments are performed on four pheromonicins of the disclosure by using three SARS-COV-2 strains (epidemic strain GD108, South Africa strain SA, and India strain IND).

The results indicate that the four pheromonicins of the disclosure have extremely significant protective efficacy against pulmonary lesions induced by the three virus strains (see Experiment 1 for details).

The disclosure also performs virus titer measurement testing on SARS-COV-2 killed by the pheromonicins, and the results indicate that the pheromonicins can effectively kill the virus and therefore reduce the live virus titer (see Experiment 2 for details).

### BRIEF DESCRIPTION OF FIGURES

FIG. 1 shows antigen target proteins on the surface of SARS-COV-2 targeted by antibody mimetics of the disclosure. Compared with the COVID-19 vaccines or drugs that have been reported or available on the market and select the S protein as a target, the disclosure selects E protein and M protein as target proteins recognized by the antibody mimetics.

FIG. 2 shows a schematic structural diagram of pheromonicins provided in the examples of the disclosure; FIG. 2a shows a pheromonicin provided in an example of the disclosure; FIG. 2b shows a pheromonicin provided in another example of the disclosure; and in the disclosure, the primary structure V_{H}CDR1-V_{H}FR2-V_{L}CDR3 of the 28-residue antibody mimetic is selected from the amino acid sequences shown in SEQ ID NO. 1 and/or SEQ ID NO. 2; FIG. 2c shows a pheromonicin provided in another example of the disclosure, the C-terminal of the pheromonicin may comprise two antibody mimetics, i.e., Ab Mimetic I and Ab Mimetic II; in specific examples, the primary structure of Ab Mimetic I may be SEQ ID NO. 1 (antibody mimetic-E) or SEQ ID NO. 2 (antibody mimetic-M), and correspondingly, the primary structure of Ab Mimetic II may be SEQ ID NO. 2 (antibody mimetic-M) or SEQ ID NO. 1 (antibody mimetic-E).

FIG. 3 shows the left lung pathological tissue sections (H. E. staining) of 30 hamsters at day 5 after challenge with the SARS-COV-2 strain GD108# in Experimental Example 1 of the disclosure, where GD108-Model refers to stained sections of the model control group, PMC-E refers to stained sections of the experimental group PMC-E, PMC-M refers to stained sections of the experimental group PMC-M, PMC-E/M refers to stained sections of the experimental group PMC-E/M, and PMC-M/E refers to stained sections of the experimental group PMC-M/E.

FIG. 4 shows a comparison chart of pathological scores of lung tissue of hamsters in different experimental groups infected with the SARS-COV-2 strain GD108# in Experimental Example 1 of the disclosure, where Model refers to the pathological score statistics of the model control group, E refers to the pathological score statistics of the experimental group PMC-E, M refers to the pathological score statistics of the experimental group PMC-M, E/M refers to the pathological score statistics of the experimental group PMC-E/M, and M/E refers to the pathological score statistics of the experimental group PMC-M/E.

FIG. 5 shows the left lung pathological sections (H. E. staining) of 30 hamsters at day 5 after challenge with the SARS-COV-2 strain SA in Experimental Example 2 of the disclosure, where SA-Model refers to stained sections of the model control group, PMC-E refers to stained sections of the experimental group PMC-E, PMC-M refers to stained sections of the experimental group PMC-M, PMC-E/M refers to stained sections of the experimental group PMC-E/M, and PMC-M/E refers to stained sections of the experimental group PMC-M/E.

FIG. 6 shows a comparison chart of pathological scores of lung tissue of hamsters in different experimental groups infected with the SARS-COV-2 strain SA in Experimental Example 2 of the disclosure, where Model refers to the pathological score statistics of the model control group, E refers to the pathological score statistics of the experimental group PMC-E, M refers to the pathological score statistics of the experimental group PMC-M, E/M refers to the pathological score statistics of the experimental group PMC-E/M, and M/E refers to the pathological score statistics of the experimental group PMC-M/E.

FIG. 7 shows the left lung pathological sections (H. E. staining) of 30 hamsters at day 5 after challenge with the SARS-COV-2 strain IND in Experimental Example 3 of the disclosure, where IND-Model refers to stained sections of the model control group, PMC-E refers to stained sections of the experimental group PMC-E, PMC-M refers to stained sections of the experimental group PMC-M, PMC-E/M refers to stained sections of the experimental group PMC-E/M, and PMC-M/E refers to stained sections of the experimental group PMC-M/E.

FIG. 8 shows a comparison chart of pathological scores of lung tissue of hamsters in different experimental groups infected with the SARS-COV-2 strain IND in Experimental Example 3 of the disclosure, where Model refers to the pathological score statistics of the model control group, E refers to the pathological score statistics of the experimental group PMC-E, M refers to the pathological score statistics of the experimental group PMC-M, E/M refers to the pathological score statistics of the experimental group PMC-E/M, and M/E refers to the pathological score statistics of the experimental group PMC-M/E.

FIG. 9 is a comparison diagram of stained pathological lung tissue sections of hamsters infected with the SARS-COV-2 strain IND in the pheromonicin PMC-E-treated experimental group and the model control group in Experimental Example 3 of the disclosure, the first line shows stained sections of the pheromonicin PMC-E-treated experimental group, and the second line shows stained sections of the model control group.

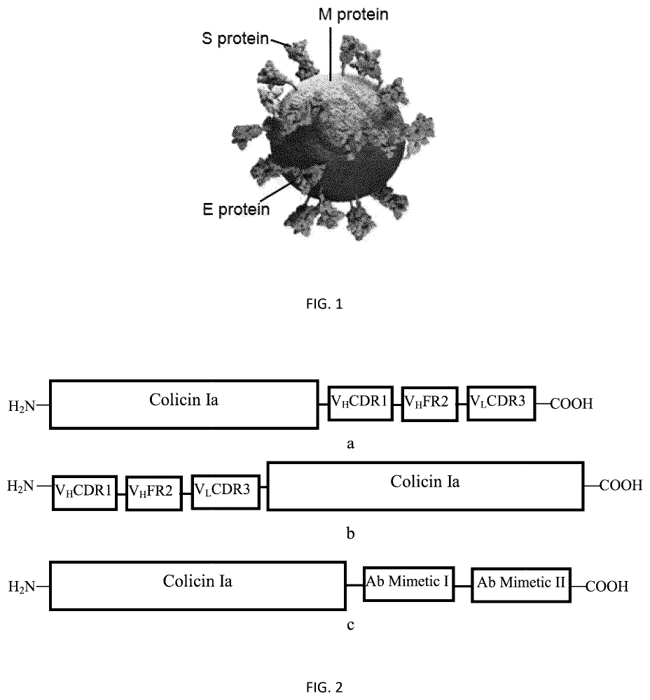

### SEQUENCE INFORMATION

SEQ ID NO: 1: The amino acid sequence of the 28-residue antibody mimetic capable of recognizing E protein
SEQ ID NO: 2: The amino acid sequence of the 28-residue antibody mimetic capable of recognizing M protein
SEQ ID NO: 3: The amino acid sequence of colicin Ia
SEQ ID NOs: 4-7: The amino acid sequences of the pheromonicin-covid-19s, i.e., pheromonicin-covid-19-E, pheromonicin-covid-19-M, pheromonicin-covid-19-E/M, and pheromonicin-covid-19-M/E, respectively.

### DETAILED DESCRIPTION

### 1. Construction of 28-Residue Antibody Mimetics Capable of Recognizing E Protein and M Protein

The full-genes synthesis of pheromonicins in plasmid construction and plasmid construction were entrusted to SinoGenoMax.

Identification: DNA sequencing of the constructed plasmid proves that the DNA sequences encoding the 28-residue antibody mimetics are located at the carboxyl terminals of the gene sequences encoding the constructed pheromonicins. LC-MS (liquid chromatography-mass spectrometry) testing of the prepared pheromonicins proves that the amino acid residues of the 28-residue antibody mimetics are located at the carboxyl terminals of the constructed pheromonicins.

### 2. Preparation of Pheromonicin-Covid-19s

The genes encoding pheromonicin-covid-19s [the gene encoding 28-residue antibody mimetic is inserted after the gene encoding the last amino acid residue 1626 of the carboxyl terminal of colicin Ia, wherein the sequence of the colicin Ia structural protein is registered in Pubmed, NCBI, number M13819.] are constructed by full-genes synthesis. The genes are inserted between the NdeI and BamHI of pET11a plasmids to form recombinant plasmids (four plasmids in total, including antibody mimetic-E, antibody mimetic-M, antibody mimetic-M/E, and antibody mimetic-E/M respectively) of the pheromonicin-covid-19s.

The plasmids are transfected into pET B-834 E. coli cells. B834 cells harboring pheromonicin-covid-19 plasmids are grown in LB liquid medium containing 100 μg/ml ampicillin and collected by centrifugation. The cells are fractured and resuspended in borate buffer (pH 9, 50 mM) and extracted to obtain supernatant by centrifugation. DNA is precipitated by adding streptomycin sulfate and supernatant is obtained by centrifugation. The supernatant is dialyzed in borate buffer and applied to DEAE agarose (Sepharose, Toyopearl SP-650-M) gel column to obtain pheromonicin-covid-19-E, pheromonicin-covid-19-M, pheromonicin-covid-19-M/E, and pheromonicin-covid-19-E/M, with a yield of 5-12 mg/ml.

### Experiment 1

### Pharmacodynamic Experiment of the Drug of the Disclosure (Pheromonicin-covid-19) Against Three SARS-COV-2 Strains

### 1. Experimental Purpose

The protective efficacy of the four pheromonicins of the disclosure against acute respiratory syndrome caused by three SARS-COV-2 strains was tested respectively.

### 2. Experimental Materials

### (1) Experimental Animals and Cells

90 male hamsters (Syrian hamsters) came from the Kunming Institute of Medical Biology, Chinese Academy of Medical Sciences.

Vero cells came from the laboratory of Sinovac.

### (2) SARS-COV-2 Strains

SARS-COV-2 epidemic strain came from Guangdong CDC, GD108 #, referred to as “GD108”.

SARS-COV-2 South Africa strain came from China CDC, DPCC-nCOV84, referred to as “SA”.

SARS-COV-2 India strain (Delta strain) came from China CDC, IND-79#, referred to as “IND”.

### (3) Drugs Under Test

The pheromonicins provided by the disclosure, i.e., the fusion proteins formed by linking the carboxyl terminal of colicin Ia to the 28-residues provided by the disclosure, were provided by Pheromonicin Biotechnology Limited, and there are four pheromonicins in total:
PMC-E: Pheromonicin-covid-19-E (colicin Ia-28-residue 1);
PMC-M: Pheromonicin-covid-19-M (colicin Ia-28-residue 2);
PMC-E/M: Pheromonicin-covid-19-E/M (colicin Ia-28-residue 1-28-residue 2); and
PMC-M/E: Pheromonicin-covid-19-M/E (colicin Ia-28-residue 2-28-residue 1).

The amino acid sequences of the 28-residue 1 and 28-residue 2 are shown in SEQ ID NO. 1 and SEQ ID NO. 2, respectively.

### 3. Experimental Grouping

Animals infected with the three SARS-COV-2 strains were divided into groups. A blank control group (control group for short) and four drug treatment groups were set for each virus strain as follows:

### (1) Groups of Syrian Hamsters Challenged With Epidemic Strain (GD108 #) for Evaluation

control group (n=6), PMC-M (n=6), PMC-E (n=6), PMC-E/M (n=6), PMC-M/E (n=6), 30 hamsters in total.

### (2) Groups of Syrian Hamsters Challenged With South Africa Strain for Evaluation

control group (n=6), PMC-M (n=6), PMC-E (n=6), PMC-E/M (n=6), PMC-M/E (n=6), 30 hamsters in total.

### (3) Groups of Syrian Hamsters Challenged With India Strain (IND) for Evaluation

control group (n=6), PMC-M (n=6), PMC-E (n=6), PMC-E/M (n=6), PMC-M/E (n=6), 30 hamsters in total.

### 4. Experimental Method

### (1) Method for Establishing Animal Models Infected With Virus

Each group of Syrian hamsters were inoculated with the three virus strains respectively via nasal drip (1×10⁵ PFU per animal).

### (2) Treatment Method and Dose

The control group was intraperitoneally injected with normal saline.

The treatment groups were intraperitoneally injected with the pheromonicins (40 mg/kg/day).

Each Syrian hamster was intraperitoneally injected with 4 mg of pheromonicins.

The above groups of animals were treated once a day and dissected post continuous treatment for 5 days.

### (3) Weight and Body Temperature Testing

Animal weight and body temperature tests were conducted 3 days before infection and the next day post treatment, both under anesthesia.

Weight was measured using an electronic digital scale.

Body temperature was measured on the abdomen using an electronic thermometer.

### (4) Specimen Treatment

The animals were anesthetized before infection and on days 1, 3, 5 and 7 post treatment to collect nasal swab specimens.

Swab specimens processing: The nasal swab specimens were lysed with 800 μl Trizol, from which 200 μl was taken to extract RNA template using an automatic nucleic acid extractor. Finally, the RNA template was dissolved in 100 μl of water and stored at −80° C. for later use. qRT PCR was performed using a one-step method.

### (5) The Animals Were Dissected on day 5 Post Treatment to Perform Gross Pathological Evaluation of Lungs With Naked Eye

Left lungs were fixed for histological sectioning, and right lungs were used for viral load measurement. Pathological observation was conducted by two pathologists with double-blind reading and scored comprehensively based on the entire pulmonary pathological map.

### (6) Statistical Analysis of Results

Individual data points were plotted for pathological scoring with prism graphed 8.0 software. Comparison of data at different time points was analyzed with paired t tests. Comparison of animal data from different groups was analyzed with unpaired t tests.

### (7) Pulmonary Pathological Scoring Criteria

According to the method reported in the article “Anti-spike IgG causes severe acute lung injury by skewing macrophage responses during acute SARS-COV infection”, pulmonary pathological changes were graded, and the scoring criteria for each indicator are shown in

Table 1 below.

TABLE 1
Score chart for pulmonary pathological
changes of hamsters in the experiment

| Score | Pathological changes |
|---|---|
| 0 | Clear alveolar structure with no inflammatory infiltration |
| 1 | Mild inflammation, slight widening of alveolar septa, and sparse |
| | mononuclear cells |
| 2 | Obvious inflammation, thickening of alveolar walls, and increased |
| | inflammatory infiltration of interstitial monocytes |
| 3-4 | Significant widening of alveolar septa and increased inflammatory |
| | cellular infiltration |
| 5 | Wide exudation and widened septa, reduced alveolar space, |
| | significant septal hemorrhage, and increased cellular infiltration in |
| | alveolar space |
| >5 | Infiltration of a large number of cells in alveolar space, |
| | disappearance of alveolar space, fusion of septa into piece, and |
| | formation of transparent membrane in alveolar wall |

According to the score chart, the comprehensive pulmonary pathological scores of respective hamsters in each group are summarized in Table 2.

TABLE 2
Individual data of pulmonary pathological scores of hamsters
in the experiment infected with three virus strains

| | Epidemic strain GD108 | | South Africa strain SA | | India strain IND | |
|---|---|---|---|---|---|---|
| | | Pathological | | Pathological | | Pathological |
| Group | Test No. | score | Test No. | score | Test No. | score |
| Model | 1 | 9 | 1 | 7 | 1 | 9 |
| | 2 | 8 | 2 | 12 | 2 | 10 |
| | 3 | 9 | 3 | 13 | 3 | 12 |
| | 4 | 7 | 4 | 12 | 4 | 13 |
| | 5 | 8 | 5 | 8 | 5 | 15 |
| | 6 | 8 | 6 | 5 | 6 | 15 |
| PMC-E | 1 | 5 | 1 | 5 | 1 | 5 |
| | 2 | 4 | 2 | 5 | 2 | 4 |
| | 3 | 3 | 3 | 5 | 3 | 5 |
| | 4 | 4 | 4 | 5 | 4 | 4 |
| | 5 | 5 | 5 | 4 | 5 | 5 |
| | 6 | 4 | 6 | 4 | 6 | 5 |
| PMC-M | 1 | 5 | 1 | 10 | 1 | 9 |
| | 2 | 5 | 2 | 4 | 2 | 10 |
| | 3 | 6 | 3 | 8 | 3 | 4 |
| | 4 | 8 | 4 | 6 | 4 | 7 |
| | 5 | 7 | 5 | 6 | 5 | 7 |
| | 6 | 6 | 6 | 6 | 6 | 5 |
| PMC-E/M | 1 | 5 | 1 | 13 | 1 | 4 |
| | 2 | 5 | 2 | 8 | 2 | 8 |
| | 3 | 4 | 3 | 12 | 3 | 7 |
| | 4 | 4 | 4 | 6 | 4 | 6 |
| | 5 | 5 | 5 | 4 | 5 | 8 |
| | 6 | 5 | 6 | 6 | 6 | 7 |
| PMC-M/E | 1 | 5 | 1 | 7 | 1 | 6 |
| | 2 | 5 | 2 | 4 | 2 | 4 |
| | 3 | 4 | 3 | 6 | 3 | 5 |
| | 4 | 4 | 4 | 5 | 4 | 7 |
| | 5 | 5 | 5 | 4 | 5 | 7 |
| | 6 | 5 | 6 | 6 | 6 | 7 |

### 5. Experimental Results

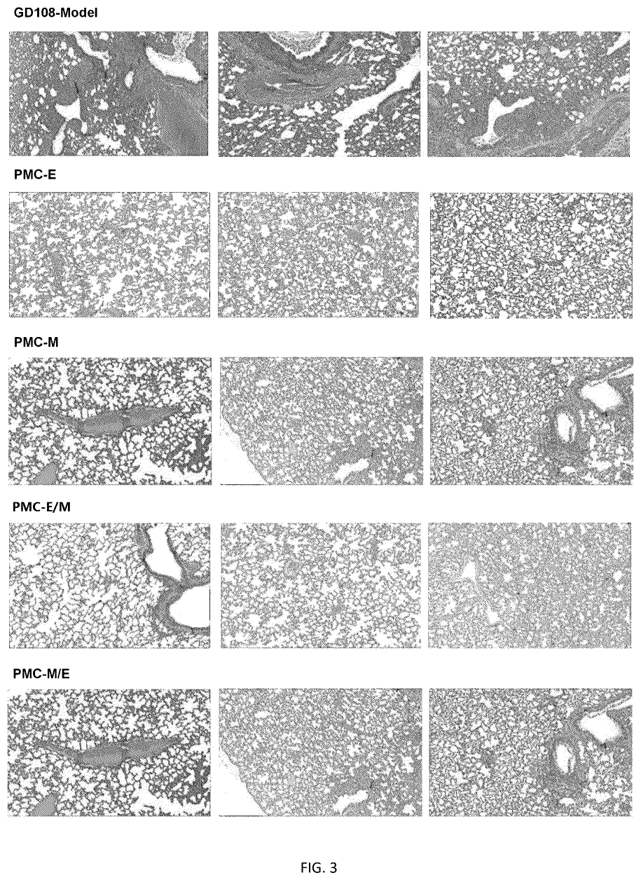

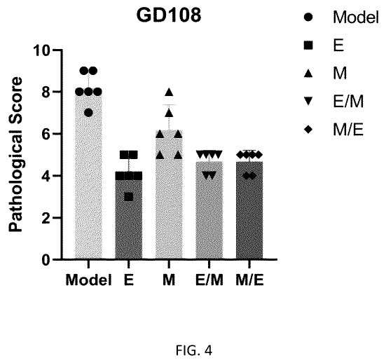

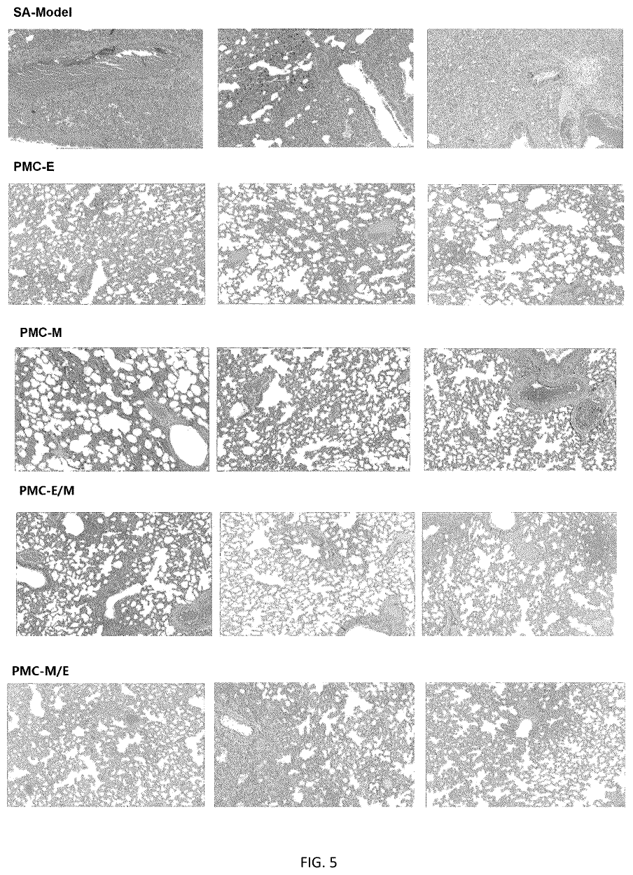

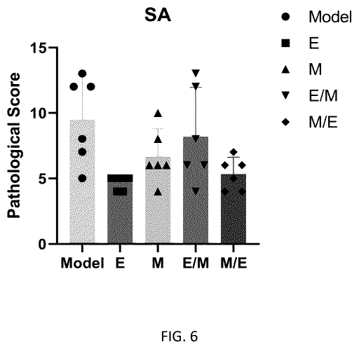

The stained pulmonary pathological sections in the experimental groups (PMC-E, PMC-M, PMC-E/M and PMC-M/E) and control group (GD108-Model) challenged with the GD108 strain are shown in FIG. 3.

The stained pulmonary pathological sections in the experimental groups (PMC-E, PMC-M, PMC-E/M and PMC-M/E) and control group (SA-Model) challenged with the SA strain are shown in FIG. 5.

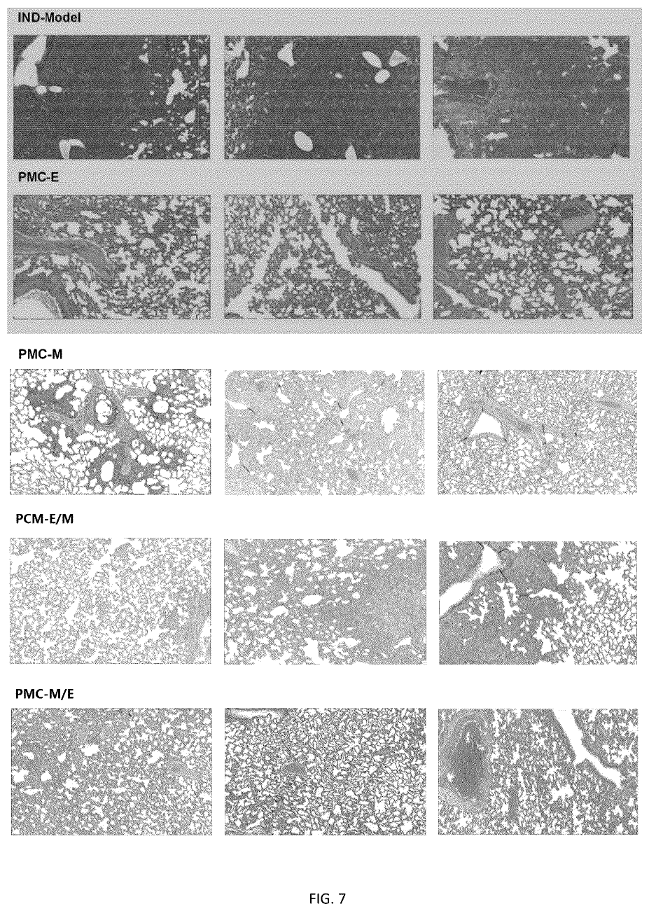

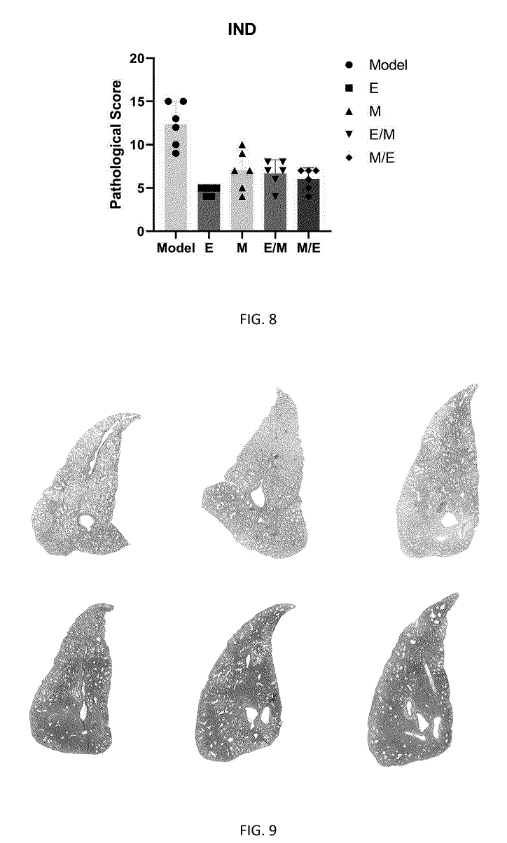

The stained pulmonary pathological sections in the experimental groups (PMC-E, PMC-M, PMC-E/M and PMC-M/E) and control group (IND-Model) challenged with the IND strain are shown in FIG. 7.

The results of the pulmonary pathological changes and the pathological scores indicated:
(1) In the SARS-COV-2 epidemic strain-infected hamster models, the drug PMC-E (p<0.0001), the drug PMC-M (p<0.01), the drug PMC-E/M (p<0.0001), and the drug PMC-M/E (p<0.0001) provided significant protective efficacy against pulmonary pathological lesions.
(2) In the SARS-COV-2 India strain (delta)-infected hamster models, the drug PMC-E (p<0.0001), the drug PMC-M (p<0.01), the drug PMC-E/M (p<0.001), and the drug PMC-M/E (p<0.001) provided significant protective efficacy against pulmonary pathological lesions.
(3) In the SARS-COV-2 South Africa strain (beta)-infected hamster models, the drug PMC-E (p<0.01) and the drug PMC-M/E (p<0.05) provided significant protective efficacy against pulmonary pathological lesions.
(4) Considering universality, for the hamster models infected with the three strains, the drug E has better protective efficacy against pulmonary pathological lesions post infection with the three strains.

### 6. Experimental Conclusion

The four drugs under test provided by the disclosure demonstrated significant protective efficacy against the three SARS-COV-2 strains.

### Experiment 2

### Virus Titer Measurement Test of the Pheromonicins of the Disclosure Against SARS-CoV-2

### 1. Experimental Purpose

The in vitro inhibitory or killing effect of the new drugs of the disclosure, i.e., pheromonicins, against SARS-COV-2 is investigated.

### 2. Experimental Materials

Drugs under test: PMC-E: pheromonicin-covid-19-E (colicin Ia—28-residue 1) and PMC-M: pheromonicin-covid-19-M (colicin Ia—28-residue 2), provided by Pheromonicin Biotechnology Limited.

The following experimental materials were provided by Beijing Sinovac Life Sciences Co., Ltd.
SARS-COV-2 epidemic strain (SARS-COV-2/human/CHN/CN1/2020, 6.0 lg CCID₅₀/ml),
Vero cells for testing,
corresponding cell culture solution.

### 3. Experimental Site

Laboratory of Beijing Sinovac Life Sciences Co., Ltd.

### 4. Experimental Method

Whether the infectivity (invasiveness) of SARS-COV-2 to cells was reduced or lost after 2-4 hours of co-incubation with the pheromonicins was tested, so as to test the inhibitory and killing effects of the pheromonicins against SARS-COV-2.

Different doses of pheromonicins (20 or 40 μg/ml culture solution) were incubated with SARS-COV-2 for 2-4 hours, and the incubated viruses were incubated with susceptible cell line cells for 24-72 hours to observe or measure cell infection and virus titer.

Based on the difference of cell infection and virus titer between the control group and other treatment groups in the test, whether the pheromonicins were effective in inhibiting and killing SARS-COV-2 was determined.

Specific operation: PMC-E and PMC-M were inoculated into cell culture plates at different dilutions, with 8 wells (50 μl/well) for each dilution. The plates were inoculated from high dilution to low dilution (10⁻⁹ to 10⁻¹). According to the Standard Operating Procedures for Resuscitation, Passage and Freezing of Vero Cells (WHO) for Testing (DX-SOP-TM-0127), Vero cells that have grown into dense monolayers were prepared into a cell suspension with a concentration of 1.00˜2.00×10⁵ cells/ml, which was then counted according to the Standard Operating Procedures for Cell Counting and Cell Vitality Testing (DX-SOP-TM-0071). The cells were added to a 96-well plate (100 μl/well), and the edge of the cell plate was gently tapped to mix the cells with the test specimen and also disperse the cells evenly. The inoculated 96-well culture plate was placed and incubated in an incubator (5% CO₂) at 36.5±1° C. for 5 days. The results were observed and recorded for 5 days.

See Table 3 for details.

TABLE 3

| | Experimental | Experimental | | | |
|---|---|---|---|---|---|
| Group | operation 1 | operation 2 | 24 h | 36 h | 72 h |
| I | Incubate the | Incubate the | Observe/ | Observe/ | Observe/ |
| | pheromonicin (20 | incubated | measure cell | measure cell | measure cell |
| | μg/ml culture | virus with | infection and | infection and | infection and |
| | solution) with the | blank cells for | virus titer | virus titer | virus titer |
| | virus for 2-4 hours | 24-72 hours | | | |
| II | Incubate the | Incubate the | Observe/ | Observe/ | Observe/ |
| | pheromonicin (40 | incubated | measure cell | measure cell | measure cell |
| | μg/ml culture | virus with | infection and | infection and | infection and |
| | solution) with the | blank cells for | virus titer | virus titer | virus titer |
| | virus for 2-4 hours | 24-72 hours | | | |
| Control | Incubate the | Incubate the | Observe/ | Observe/ | Observe/ |
| | culture solution | incubated | measure cell | measure cell | measure cell |
| | (same volume as | virus with | infection and | infection and | infection and |
| | pheromonicin) | blank cells for | virus titer | virus titer | virus titer |
| | with the virus for | 24-72 hours | | | |
| | 2-4 hours | | | | |

### 5. Experimental Results

After incubation of the drugs under test, i.e., two pheromonicins, with the viruses for 3 hours, all the live virus titers were reduced by 1.5 lgCCID₅₀/ml compared to that of the control group (the live virus titer of the control group was 8 lgCCID₅₀/ml after 3 hours of incubation). The results are shown in Table 4.

TABLE 4

| Name of test | Dilution | | | | | | | | | | |
|---|---|---|---|---|---|---|---|---|---|---|---|
| specimen | 10⁻¹ | 10⁻² | 10⁻³ | 10⁻⁴ | 10⁻⁵ | 10⁻⁶ | 10⁻⁷ | 10⁻⁸ | 10⁻⁹ | IgCCID₅₀/0.05 ml | IgCCID₅₀/ml |
| PMC-E treatment | 8/8 | 8/8 | 8/8 | 8/8 | 6/8 | 0/8 | 0/8 | 0/8 | 0/8 | 5.25 | 6.5 |
| group | | | | | | | | | | | |
| PMC-E control | 8/8 | 8/8 | 8/8 | 8/8 | 8/8 | 8/8 | 1/8 | 1/8 | 0/8 | 6.75 | 8.0 |
| group | | | | | | | | | | | |
| PMC-M treatment | 8/8 | 8/8 | 8/8 | 8/8 | 6/8 | 0/8 | 0/8 | 0/8 | 0/8 | 5.25 | 6.5 |
| group | | | | | | | | | | | |
| PMC-M control | 8/8 | 8/8 | 8/8 | 8/8 | 8/8 | 8/8 | 2/8 | 0/8 | 0/8 | 6.75 | 8.0 |
| group | | | | | | | | | | | |

### 6. Experimental Conclusion

The pheromonicins of the disclosure have inhibitory and killing effects against SARS-CoV-2.

1. Polypeptides with amino acid sequences shown in SEQ ID NO. 1 and/or SEQ ID NO. 2.

2. Use of the polypeptides with amino acid sequences shown in SEQ ID NO. 1 and/or SEQ ID NO. 2 in preparation of drugs against SARS-COV-2.

3. The use of claim 2, wherein the drugs comprise preparations for preventing, treating and diagnosing SARS-COV-2.

4. Drugs against SARS-COV-2, comprising fusion proteins obtained by linking channel-forming E1 family colicins to the polypeptides with the sequences shown in SEQ ID NO. 1 and/or SEQ ID NO. 2.

5. The drugs of claim 4, wherein the channel-forming E1 family colicins are selected from colicins E1, Ia, Ib, A, B, or N.

6. The drugs of claim 5, wherein the channel-forming E1 family colicin is colicin Ia with an amino acid sequence shown in SEQ ID NO. 3.

7. The drugs of claim 4, wherein the linkage is achieved by linking the polypeptides with the sequences shown in SEQ ID NO. 1 and/or SEQ ID NO. 2 to the carboxyl terminal and/or amino terminal of the colicin.

8. The drugs of claim 7, wherein the linkage is achieved by linking the polypeptides with the sequences shown in SEQ ID NO. 1 and/or SEQ ID NO. 2 to the carboxyl terminal of the colicin.

9. The drugs of claim 8, wherein the fusion proteins have a polypeptide arrangement selected from the following sequences:
SEQ ID NO. 3-SEQ ID NO. 1;
SEQ ID NO. 3-SEQ ID NO. 2;
SEQ ID NO. 3-SEQ ID NO. 1-SEQ ID NO. 2; or
SEQ ID NO. 3-SEQ ID NO. 2-SEQ ID NO. 1.

10. A method for preparing drugs against SARS-COV-2, wherein polypeptides with amino acid sequences shown in SEQ ID NO. 1 and/or SEQ ID NO. 2 are linked to colicin.

11. The drugs of claim 5, wherein the linkage is achieved by linking the polypeptides with the sequences shown in SEQ ID NO. 1 and/or SEQ ID NO. 2 to the carboxyl terminal and/or amino terminal of the colicin.

12. The drugs of claim 6, wherein the linkage is achieved by linking the polypeptides with the sequences shown in SEQ ID NO. 1 and/or SEQ ID NO. 2 to the carboxyl terminal and/or amino terminal of the colicin.
